(19) United States
(12) Patent Application Publication
(10) Pub. No.: US 2012/0220541 A1
(43) Pub. Date: Aug. 30, 2012

(54) USE OF YEAST PEPTIDE HYDROLYSATE AS AN ACTIVE AGENT FOR STRENGTHENING HAIR

(75) Inventors: Claude Dal Farra, Kerhonkson, NY (US); Nouha Domloge, Valbonne (FR); Jean-Marie Botto, Valbonne (FR)

(73) Assignee: ISP INVESTMENTS INC., Wilmington, DE (US)

(21) Appl. No.: 13/505,348

(22) PCT Filed: Nov. 2, 2010
(86) PCT No.: PCT/FR10/00724

§ 371 Date: May 1, 2012

(30) Foreign Application Priority Data
Nov. 3, 2009 (FR) 0905257

(51) Int. Cl. A61K 8/64; A61Q 5/00

(52) U.S. Cl. 514/20.7

(57) ABSTRACT

The invention concerns the use of a composition comprising at least one yeast (Saccharomyces cerevisiae) peptide hydrolysate as an active agent for strengthening hair and improving hair health. The invention also concerns the use of this novel active agent for making a dermo-pharmaceutical composition intended to stimulate hair growth or to prevent hair loss. The invention furthermore refers to a cosmetic treatment method intended to stimulate hair growth or counteract hair loss and counteract external aggressions affecting the hair.

### FIELD OF THE INVENTION

The present invention is in the cosmetic and dermo-pharmaceutical fields. The invention concerns the use of a composition comprising at least one yeast (Saccharomyces cerevisiae) peptide hydrolysate as an active agent for strengthening hair and improving hair health. The invention also concerns the use of this novel active agent for making a dermo-pharmaceutical composition intended to stimulate hair growth or to prevent hair loss as part of a preventive or curative treatment for hair loss related to a pathological condition.

The invention further relates to a cosmetic treatment method designed to stimulate hair growth or prevent hair loss and to prevent external aggressions which affect hair, in which a composition containing the active agent is applied topically to the treatment area.

The invention further relates to a cosmetic treatment method intended to prevent or counteract external aggressions.

### BACKGROUND OF THE INVENTION

Hair growth and hair renewal are primarily determined by the activity of hair follicles and their matrix environment.

The hair follicle is a complex independent skin adjunct which comprises six major compartments, some of dermal origin (conjunctive tissue sheath and dermal papilla) and some of epithelial origin (internal and external epithelial root sheathes, hair shaft and sebaceous stem). At the base of the follicle is found the highly vascularized matrix, which is the origin of the three concentric layers of the hair. The hair consists of differentiated epithelial cells essentially containing keratins, which are in turn organized according to very stable protein superstructures. The organization and strength of the hair shaft and hair are determined by the activity and health of the follicle.

The hair follicle is renewed according to a cycle consisting of three phases: the anagen phase, the catagen phase, and the telogen phase.

The anagen phase (growth phase) lasts between one and ten years and is characterized by a constant lengthening of the hair. At the beginning of each anagen phase, development of the perifollicular microvascular network is observed. Perifollicular microcirculation therefore plays a fundamental role in the hair growth process by providing factors and nutrients needed for follicle growth.

The catagen phase which follows only lasts a few weeks. During this phase, the blood capillaries collapse and disappear. The follicle atrophies and withdraws to the surface, with the noteworthy exception of the dermal papilla, which will be the key element of future regeneration.

The terminal or telogen phase, which lasts a few months, is a rest phase for the follicle, after which the hair ends up dropping. At the end of this rest period, a new follicle is regenerated and a new cycle begins.

Furthermore, it has been well documented that the differentiation mechanisms of keratinocytes of the epidermis and hair follicle are substantially different. For instance, it is known that hair shaft keratins are a family of keratins separate from that expressed in the epidermis (Langbein et al., 2001, J. Biol. Chem. 276); for instance, keratin K6irs (Door et al., 2001, Rb. J. Dermatol. 145: 558-568) is expressed in the internal sheath of the hair follicle, but not in the epidermis, whereas the epidermal differentiation markers, such as keratins K1 and K10, are not expressed in the hair follicle (Lenoir et al., 1988, Dev. Biol. 130: 610-620).

Natural dropping or loss of hair can be estimated at about one hundred hairs, on average, per day for a normal physiological condition. This ongoing renewal process can be disrupted by numerous intrinsic and extrinsic factors resulting in substantial hair loss, be it temporary or not, which come under the general term of alopecia. For instance, microcirculation disturbances have been observed in alopecic skin. At the same time, a kind of perifollicular fibrosis takes root in alopecic areas, the follicles shrink from one cycle to the next, and vascularization of the bulbs progressively dwindles.

In addition, the ongoing physiological hair renewal process is subject to aging. While the most visible sign of hair aging is graying, the quality of the hair follicle's biological environment is also affected. Among the manifestations of hair aging we find diminished synthesis of the extracellular matrix proteins (collagen, laminin, fibronectin), resulting in a loss of elasticity and tonus of the subcutaneous tissues. There is also a drop in the coherence and organization of hair follicles, a decrease in the duration of the anagen phase, and a lengthening of the duration of the telogen phase (Courtois et al., 1995, Br. J. dermatol., 132: 86-93). In effect, the hair loses its elasticity and becomes thinner and therefore more fragile. Considering the scalp as a whole, aging is evidenced by a drop in capillary density and by a gradual decrease in the diameter of the follicles, giving the hair a thinner, more sparse appearance (Pelfini, C. et al., J. Méd. Esth. Et Chir. Derm.1987; Birch M P et al. Br. J. Dermatol 2001; 144: 297-304).

Independently of intrinsic aging, alterations of the hair or hair follicle may occur as a result of external aggressions. Indeed, although hair is remarkably stable, certain external factors such as the sun, which causes photo-aging, free radicals, pollution, or certain inappropriate treatments, can lead to premature damage to the structure of hair and follicle depletion.

The expression “external aggression” is understood to mean aggressions which the environment can produce. As an example, let us mention aggressions such as pollution, ultraviolet light, or irritating products such as excessively detersive surfactants, dyes and bleaches, excessively frequent permanents or straightening, mechanical aggressions such as rubbing by clothing, hair styling causing repeated stretching, overly intense brushing, teasing, and blow-drying with air that is too hot. Pollution is understood as both “external” pollution due, for example, to diesel particles, ozone, or heavy metals, and “internal” pollution, which may be due, for instance, to solvent emissions from paints, glues, or wallpaper (such as toluene, styrene, xylene, or benzaldehyde), or cigarette smoke. These external aggressions lead to an alteration of the external hair structure and the hair's mechanical properties, but can also affect the hair follicle and cause premature aging.

The cosmetic or pharmaceutical industry is always looking for compositions which can eliminate or reduce hair loss or stimulate hair growth. Worth mentioning is the known molecule 2,4-diamino-6-piperidinopyrimidine-3-oxide or “Minoxidil” (U.S. Pat. Nos. 4,139,619 and 4,596,812). A known compound for maintaining perifollicular vascularization is 4-verapamil, described as being active in the treatment of hair loss, particularly through their effects on microcirculation (JP 88/062680).

Furthermore, there are other patents describing the use of topical vasodilators intended to stimulate hair growth by acting on the microcirculation of the scalp (EP 327 263). Another patent document by Shiseido (JP 07316023) also describes the use of arginin and its derivatives in the treatment of alopecia. However, some available products have side effects, such as in the case of Minoxidil, or have a limited effectiveness that lasts only as long as the treatment. Consequently, there is a need for a new, physiologically acceptable composition for promoting hair growth and/or reducing hair loss which acts quickly and is effective over the long term.

Yeast peptide extracts have previously been described for their hair fiber hydration properties for use in hair conditioner compositions (EP0695801).

In addition, yeast peptide extracts have previously been described for their effects on skin (FR 2904 552, FR 2887 772). The inventors have now shown that such yeast peptide hydrolysates had an action in strengthening the hair follicle structure and improving hair health. In particular, it has been demonstrated that the peptide hydrolysate, when applied to hair, increases differentiation of the epithelial cells in the external sheath of the follicle, improves vascularization of the follicle, and increases the expression of extracellular matrix proteins.

### DISCLOSURE OF THE INVENTION

The first subject matter of the invention is the cosmetic use of a composition comprising at least one yeast (Saccharomyces cerevisiae) peptide hydrolysate as an active agent for strengthening hair and improving hair health.

The human keratin fibers to which the invention applies are hair, eyebrows, eyelashes, facial hair, public hair, and nails. More specifically, the invention applies to human hair and/or eyelashes.

A “peptide hydrolysate” is understood as a mixture of compounds consisting for the most part of peptides or oligopeptides. According to the invention, the terms “peptide hydrolysate” and “active agent” will be used indifferently.

The phrase “active agent capable of strengthening hair and improving hair health” refers to any peptide hydrolysate coming from yeast which is capable of increasing the expression of keratins K14 and K17, increasing the expression of extracellular matrix proteins such as collagen I, and increasing the expression of basal plate proteins such as collagen IV.

It is also understood that the active agent is capable of increasing the expression of proteins present in the wall of blood vessels in the follicle, such as protein CD34 and collagen IV.

The active agent according to the invention can be made by extraction of yeast proteins followed by controlled hydrolysis which releases compounds having a peptide nature.

“Compounds having a peptide nature” are understood as the protein fragments and peptides present in the peptide hydrolysate of the invention.

The use of peptide hydrolysates, and particularly low-molecular-weight peptide hydrolysates, offers numerous benefits in cosmetics. In addition to generating compounds having a peptide nature which did not exist in the initial protein mixture, hydrolysis and purification make it possible to obtain more stable mixtures which are more easily standardized and do not trigger allergic reactions in dermo-cosmetics.

The hydrolysate from the yeast genus Saccharomyces must be understood as a yeast hydrolysate belonging to the genus Saccharomyces. Naturally, the hydrolysate can be prepared from yeast from at least any one of the numerous varieties and species belonging to the genus Saccharomyces. According to a preferred embodiment, said active agent comes from the hydrolysis of yeast proteins from the species Saccharomyces cerevisiae.

A great number of yeast proteins are likely to contain bioactive peptides in their structure. Controlled hydrolysis makes it possible to bring out these compounds having a peptide nature. It is possible, but not necessary for the purposes of the invention, to extract the proteins in question first and to hydrolysate them after, or to first conduct hydrolysis of the raw extract and subsequently to purify the compounds having a peptide nature.

According to a currently preferred method of the invention, the yeast hydrolysate is an aqueous extract. An aqueous extract is understood as any combination of compounds soluble in water or in any solvent consisting entirely or partially of water. The extracts of the invention are namely purified aqueous extracts. In particular, aqueous solvents may be cited. An aqueous solvent is understood as any solvent consisting entirely or partially of water. Let us mention water itself, glycerol, water-and-alcohol solvents in any proportion, or solvents consisting of water and a compound such as propylene glycol or butylene glycol in any proportion. This hydrolysate can be obtained by dissolving in water, alcohol, or ether, then concentrating the solution by means of evaporation or distillation.

Any extraction or purification method known to a person skilled in the art can be used to prepare the hydrolysate of the invention.

For instance, in the first step, the yeasts are cultured in a conventional manner in a medium appropriate for their development, preferably in the presence of lactose. They are collected by centrifugation and then suspended in a buffer solution, preferably a phosphate buffer. In a second step, these cells are disrupted by means of a French press or a ball mill, with most of the insoluble membrane components being eliminated by centrifugation or filtration.

A protein-rich filtrate can then be collected and re-dissolved. A fraction rich in compounds having a peptide nature can then be isolated by precipitation in an alcohol medium or with a saline solution. The soluble components and nucleic acids are thus eliminated. According to a variant of the method for producing the hydrolysate of the invention, a dialysis step and a hydrolysis step using a cocktail of proteases can be added to obtain a fraction rich in compounds having a peptide nature.

An additional purification step using a chromatography method can be considered.

According to another method for producing the yeast hydrolysate of the invention, the hydrolysis step can be performed directly on the yeasts collected after centrifugation or on the fractions obtained after cell disruption. Filtration and sterilization steps are then performed.

A phase of dilution in water or any mixture containing water is performed, then the dilution is sterilized by ultrafiltration in order to obtain a peptide hydrolysate having a protein concentration of 30% and 70% of the total dry extract weight, with this concentration more specifically being 40% and 50% of the total dry extract weight. The solvents used are physiologically acceptable and conventionally used by a person skilled in the art, and are chosen from glycerol, ethanol, propanediol, butylene glycerol, the dipropylene glycerol, ethoxylated or propoxylated diglycols, cyclic polyhydric alcohols, or any mixture of these solvents.

In this way, the active agent of the invention is advantageously dissolved in one or more physiologically acceptable solvents, such as water, glycerol, ethanol, propanediol, butylene glycol, dipropylene glycerol, ethoxylated or propoxylated diglycols, cyclic polyhydric alcohols, or any mixture of these solvents. The diluted active agent is then sterilized by ultrafiltration.

After this dilution step, the active agent can be encapsulated or included in a cosmetic or pharmaceutical vector such as liposomes or any other microcapsule used in the field of cosmetics or adsorbed in powdered organic polymers, mineral substrates such as talcs or bentonites.

According to an advantageous embodiment, the active agent is present in the compositions of the invention at a concentration of between approximately 0.001% and 5%, and preferably at a concentration of between approximately 0.01% and 1% in relation to the total weight of the final composition.

The compositions which can be used according to the invention may be in the form of an aqueous, water-and-alcohol, or oil solution; an oil-in-water emulsion, a water-in-oil emulsion, or multiple emulsions; they may also be in the form of crèmes, suspensions, or powders. These compositions can be more or less fluid and have the appearance of a crème, lotion, salve, serum, ointment, gel, paste, or foam. They may also be in solid form, such as a stick, or be applied to the area requiring treatment in aerosol form. They may be used as a care product and/or as a skin make-up product.

All of these compositions furthermore include any additive customarily used in the field of application under consideration, as well as the additives needed for their formulation, such as co-solvents (ethanol, glycerol, benzyl alcohol, moisturizers, etc.), thickeners, thinners, emulsifiers, antioxidants, dyes, sunscreens, pigments, fillers, preservatives, scents, odor absorbers, essential oils, trace elements, essential fatty acids, surfactants, film-forming polymers, chemical or mineral filters, hydrating agents, or thermal spring water, etc. Let us mention, for example, natural water-soluble polymers such as polysaccharides, polypeptides, cellulose derivatives such as methylcellulose or hydroxypropyl cellulose, or synthetic polymers, poloxamers, carbomers, PVA or PVP, and particularly polymers sold by the company ISP.

In any event, a person skilled in the art will ensure that these additives and their proportions are chosen in such a way as not to harm the desired advantageous properties of the composition of the invention. For example, these additives may account for a concentration of between 0.01% and 20% of the total weight of the composition. When the composition of the invention is an emulsion, the fatty phase may account for 5% to 80% by weight, and preferably 5% to 50% by weight, in relation to the total weight of the composition. The emulsifiers and co-emulsifiers used in the composition are to be chosen from those conventionally used in the field under consideration. For example, they may be used in a proportion of between 0.3% to 30% by weight, in relation to the total weight of the composition.

The compositions which can be used according to the invention may consist of shampoos, conditions, a treatment lotion before or after aggressive hair treatments, a hair crème or gel, a hair restructuring lotion, a mask, etc. The composition may also be in the form of mascara for application to eyelashes, eyebrows, or hair.

Furthermore, the active agent of the invention may be used alone or in combination with other active agents.

Advantageously, the compositions which can be used according to the invention additionally contain at least one other active agent which protects or improves hair growth and/or health. Let us mention, as non-limiting examples, the following ingredients: vitamins, anti-free radical and anti-UV agents, other plant-based peptide extracts, minoxidil, nicotinic acid esters, anti-inflammatory agents, retinoic acid or its derivatives, retinol, 5-alpha reductase inhibitors, or chemically-synthesized peptide compounds.

The composition which can be used according to the invention can be applied by any appropriate means, such as orally, parenterally, or topically, and the formulation of the compositions shall be adapted by a person skilled in the art, particularly for cosmetic or dermatological compositions.

Advantageously, the compositions of the invention are intended for topical administration. These compositions must therefore contain a physiologically-acceptable medium, that is, one compatible with the skin and keratinous appendages, and encompassing all cosmetic or dermatological forms.

“Topical application” is understood as the act of applying or spreading the active agent of the invention, or a composition containing said agent, to the surface of the skin or a mucous membrane. “Physiologically acceptable” means that the peptide hydrolysate of the invention, or a composition containing said hydrolysate, is appropriate for coming into contact with the skin or a mucous membrane without causing toxicity or intolerance reactions.

According to another aspect of the invention, the composition containing the yeast (Saccharomyces cerevisiae) peptide hydrolysate as the active agent is used to treat alopecia.

Alopecia encompasses a range of hair follicle problems which ultimately result in temporary or permanent, partial or general hair loss. Both men and women can be affected by alopecia, but the areas predominantly affected in men are the temples or forehead, whereas in women there is a diffuse alopecia of the vertex.

The second subject matter of the invention is the use of a yeast peptide hydrolysate (Saccharomyces cerevisiae) as an active agent for strengthening the hair follicle structures. On the histological level, this action is characterized by increased differentiation of the epithelial cells in the external sheath of the follicle, stimulated vascularization of the hair follicle, and increased density of the extracellular matrix and the basement membrane.

On the molecular level, the action of the yeast (Saccharomyces cerevisiae) peptide hydrolysate of the invention is characterized by increased expression of keratins K14 and K17.

The action of the yeast (Saccharomyces cerevisiae) peptide hydrolysate of the invention is also characterized by increased expression of protein CD34 and collagen IV in the wall of hair follicle blood vessels.

The action of the yeast (Saccharomyces cerevisiae) peptide hydrolysate of the invention is further characterized by increased expression of collagen I in the extracellular matrix and increased expression of collagen IV in the basement membrane of the hair follicle.

The third subject matter of the invention refers to a cosmetic treatment method intended to prevent or counteract external aggressions against hair, characterized in that the composition of the invention is applied topically to the area being treated.

The fourth subject matter of the invention refers to a cosmetic treatment method intended to restore and/or stimulate hair growth or counteract hair loss, characterized in that the composition of the invention is applied topically to the area being treated. According to a special embodiment, the invention refers to a cosmetic treatment method intended to restore and/or stimulate eyelash growth or counteract eyelash loss, characterized in that the composition of the invention is applied topically to the area being treated

The invention also refers to the use of a yeast (Saccharomyces cerevisiae) peptide hydrolysate as an active agent capable of strengthening and protecting the hair follicle for making a dermo-pharmaceutical composition intended to stimulate hair growth or to counteract hair loss as part of a preventive or curative treatment for hair loss related to a pathological condition. Of the pathological conditions frequently responsible for hair loss, let us mention alopecia areata, the side effects of drug treatments, and certain infections or inflammations of the scalp (psoriasis, seborrheic dermatitis, etc.).

According to this embodiment of the invention, the compositions are appropriate for oral administration for pharmaceutical use. For instance, the compositions can be in the form of tablets, capsules, gelcaps, chewing paste, powders to be ingested as is or to be mixed extemporaneously with a liquid, syrup, or gel, and any other form known to a person skilled in the art. These compositions furthermore include any additive customarily used in the field of application under consideration, as well as any additives required for their formulation, such as solvents, thickeners, thinners, antioxidants, preservatives, other pharmaceutical active agents, essential oils, vitamins, essential fatty acids, etc.

The invention furthermore refers to a cosmetic treatment method intended to prevent or counteract the signs of aging and photo-aging of the hair, characterized in that the composition of the invention is applied topically to the area being treated.

The invention furthermore refers to a cosmetic treatment method intended to stimulate nail growth, characterized in that the composition of the invention is applied topically to the area being treated.

Particular embodiments of this cosmetic treatment method also arise from the foregoing description. Other advantages and features of the invention will become more readily apparent upon reading the examples, which are given solely for non-limiting, illustration purposes.

### LIST OF FIGURES

FIG. 1: Immunolabeling of keratin K14 in the hair follicle treated with the hydrolysate according to example 1.

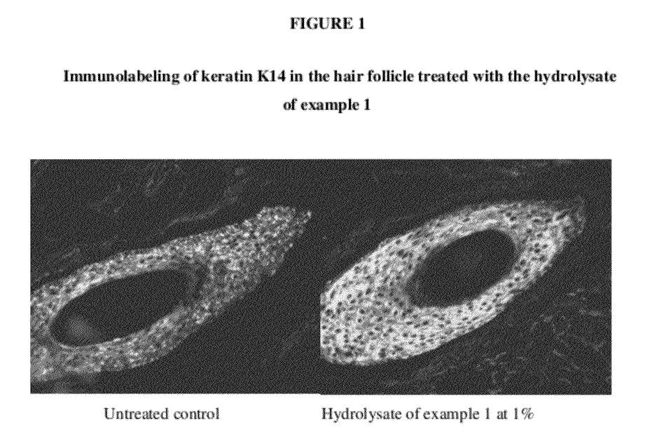

### EXAMPLE 1

### Preparation of a Yeast (Saccharomyces cerevisiae) Peptide Hydrolysate

The active agent is obtained from a yeast extract from the species Saccharomyces cerevisiae. The yeasts are cultured in a medium suitable for their development, then centrifuged to recover a biomass.

The biomass is then milled in a ball mill Next, the ground material is re-dissolved in water at a concentration of 100 grams per liter, before enzymatic hydrolysis at between 40% and 60° C. for 6 hours. After hydrolysis, the extract is centrifuged and then diluted in a water-glycerol mixture. The extract is then filtered before sterilization.

A hydrolysate containing a quantity of protein and peptide compounds accounting for approximately 30% to 70% of the total weight of the dry extract is obtained, with this quantity especially being between 40% and 50% of the total weight of the dry extract.

A determination of the amino acid composition of the active agent of the invention was also performed. After assaying the proteins and peptides using the Lowry method, acid hydrolysis was performed to reduce all the peptides to the state of free amino acids. An example of the amino acid composition of the hydrolysate is given in the following table. The values are expressed in percentage of amino acids per 100 g of proteins.

| Amino acids | % |
|---|---|
| Alanine | 7.4 |
| Aspartic acid | 12.0 |
| Arginine | 4.6 |
| Glutamic acid | 15.6 |
| Glycine | 5.5 |
| Histidine | 2.8 |
| Isoleucine | 4.6 |
| Leucine | 9.2 |
| Lysine | 8.3 |
| Phenylalanine | 5.5 |
| Proline | 4.6 |
| Serine | 6.5 |
| Threonine | 5.5 |
| Tyrosine | 4.6 |
| Valine | 6.5 |
| Tryptophan | ND |

### EXAMPLE 2

### Demonstration of Stimulated Differentiation of the Epithelial Cells in the External Sheath of the Hair by the Hydrolysate of Example 1

The purpose of this study is to determine the influence of the hydrolysate of example 1 on the differentiation of epithelial cells in the external sheath of the hair. To do this, keratinocyte differentiation markers were studied. Keratin K14 is an early differentiation marker expressed in the proliferative compartment of the external epithelial root sheath of the hair follicle. Keratin K17 is late differentiation marker closely associated with the strength of the hair shaft.

Protocol: Skin biopsies measuring 6 mm in diameter, coming from face lifts and containing hair follicles, were cultured on inserts in William's E medium and then treated for 48 hours with the hydrolysate of example 1 at 1%. Untreated controls were also made. At the end of the experiment, the biopsies were placed in cassettes and dipped into a 10% mixture of formaldehyde for 2 hours in an automated instrument (VIP). The paraffin coating was prepared by a series of alcohol baths (with increasing concentrations and times), followed by 2 xylene baths and lastly a paraffin bath. The total duration of this series of operations was about 12 hours. The biopsies included in paraffin were then cut to 4 μm by a microtome and placed on slides. The slides are deparaffinized, rehydrated, and then subjected to immunolabeling with a monoclonal antibody directed against keratin K14 (Abcam) or a monoclonal antibody directed against keratin K17 (Abcam), then a second appropriate antibody coupled to a fluorescent marker. The skin slices are then examined with an Epi-fluorescence microscope (Nikon Eclipse E 80i microscope).

Results: A much more intense fluorescence of the external epithelial root sheath cells is observed in the hair follicle slices treated with the hydrolysate according to example 1 and marked for keratin K14 or keratin K17, compared to the untreated control.

Using fluorescence quantification software, a 44% increase for keratin K14 and a 44% increase for keratin K17 can be measured.

Conclusions: The hydrolysate of example 1 increases keratinocyte differentiation, particularly for keratin K17 which is closely associated with hair shaft strength. The hydrolysate of example 1 increases the cohesion of the epithelial root sheath and improves the conformation of the internal epithelial root sheath.

On the whole, this study demonstrates that the active agent of the invention strengthens hair structure.

### EXAMPLE 3

### Demonstration of the Stimulating Effect of the Hydrolysate of Example 1 on Hair Follicle Vascularization

The purpose of this study is to determine the influence of the hydrolysate of example 1 on the hair follicle blood vessels. To do this, the expression of blood vessel wall markers was studied.

Protocol: The cultures and paraffin inclusions were made according to the same protocol as example 2. The slides were deparaffinized, rehydrated, and then subjected to an unmasking step and then immunolabeling with a monoclonal antibody directed against collagen IV (Chemicon) or against protein CD34 (Novocastra), then an adapted secondary antibody coupled to a fluorescent marker. The skin slices were then examined with an Epi-fluorescence microscope (Nikon Eclipse E 80i microscope).

Results: A more intense fluorescence of the basal plate and the blood vessel wall is observed in the hair follicle slices treated with the hydrolysate of example 1, in which the collagen IV was immunolabeled, compared to the untreated control. Using fluorescence quantification software, a 144% increase can be measured.

A more intense fluorescence of the blood vessel wall and the conjunctive tissue sheath is observed in the hair follicle slices treated with the hydrolysate of example 1, in which protein CD34 was immunolabeled, compared to the untreated control.

Conclusions: The hydrolysate of example 1 improves the blood circulation of the follicle, which has the result of increasing the supply of nutrients and thus improving hair health.

In addition, the hydrolysate of example 1 improves the hair follicle structures.

### EXAMPLE 4

### Demonstrating the Strengthening of Matrix Structures by the Hydrolysate of Example 1

The purpose of this study is to determine the influence of the hydrolysate of example 1 on the extracellular matrix and the dermal papilla of the follicle. To do this, the expression of one of the primary proteins of the matrix was studied.

Protocol: The cultures and paraffin inclusions were made according to the same protocol as example 2. The slides were deparaffinized, rehydrated, and then subjected to an unmasking step and then immunolabeling with a monoclonal antibody directed against collagen I (Tebu-Rockland), then an adapted secondary antibody coupled to a fluorescent marker. The skin slices were then examined with an Epi-fluorescence microscope (Nikon Eclipse E 80i microscope).

Results: A more intense fluorescence of the conductive tissue sheath and the dermal papilla is observed in the hair follicle slices treated with the hydrolysate of example 1, in which the collagen I was immunolabeled, compared to the untreated control.

Conclusions: The hydrolysate of example 1 strengthens the dermal part surrounding the follicle and thus provides better cohesion and better protection of the hair follicle structure.

### EXAMPLE 5

### Preparation of Compositions

1—Nourishing Treatment for Hair and Scalp

Apply the product to the wet scalp. Massage to spread the product uniformly. Strengthens hair while making it smooth and easy to style.

| | | Formulation | | |
|---|---|---|---|---|
| | | 1 | 2 | |
| | | % by | % by | |
| INCI Name | Trade name | weight | weight | Supplier |
| Phase A | | | | |
| Deionized Water | — | Q.S. | Q.S. | |
| Aminomethyl | AMP-95 | 0.05 | 0.05 | |
| Propanol | | | | |
| Acrylic Acid/VP | UltraThix ™ | 0.85 | 0.85 | ISP |
| Crosspolymer | P-100 | | | |
| Phase B | | | | |
| Gycerol Dilaurate | Emulsynt ™ | 0.50 | 0.50 | ISP |
| | GDL | | | |
| Jojoba Seed Oil | — | 2.00 | 2.00 | Lipo |
| Cetearyl Alcohols | — | 2.00 | 2.00 | Rita |
| Phase C | | | | |
| Cyclopentasiloxane | SiTec ™ | 0.50 | 1.00 | ISP |
| | CM040 | | | |
| Phase D | | | | |
| VP/DMAPA Acrylates | Styleze ® | 3.00 | 3.00 | ISP |
| Copolymer | CC-10 | | | |
| Water | | 20.00 | 20.00 | |
| Aminomethyl | | 0.37 | 0.37 | |
| Propanol | | | | |
| Phase E | | | | |
| Diazolidinyl Urea | Germaben ® | 0.75 | 0.75 | ISP |
| (and) Methylparaben | M | | | |
| (and) Propylene | | | | |
| Glycol | | | | |
| | Hydrolysate of | 0.50 | 1.00 | ISP |
| | example 1 | | | |
| Total | | 100.00 | 100.00 | |

Put the water and AMP-95 in a container with stirring. Add the UltraThix™ P-100 to the water with vigorous stirring and keep stirring for 30 minutes. Heat phase A to 65° C. Heat the phase B ingredients to 65° C. then mix them. Add to phase B and mix carefully. Cool to 35° C. Add phase C to the primary mixture and mix until a uniform appearance is obtained.

Separately, mix the ingredients of phase D until a uniform appearance is obtained. Add the Germaben®M (Phase E) and mix until a uniform appearance is obtained. Add the hydrolysate of example 1 and stir until a uniform appearance is obtained.

2—Hair Growth Serum:

Apply the product to the wet scalp. Massage to spread the product uniformly. Promotes hair growth or regrowth and makes hair stronger.

| | | Formulation | | |
|---|---|---|---|---|
| | | 1 | 2 | |
| | | % by | % by | |
| INCI Name | Trade name | weight | weight | Supplier |
| Water | | Q.S. | Q.S. | |
| Hydroxyethyl- | Natrosol | 0.35 | 0.50 | Hercules/ |
| cellulose | 250HHR | | | Aqualon |
| Disodium EDTA | Dissolvine | 0.05 | 0.05 | Akzo Nobel |
| | NA-2S | | | |
| VP/DMAPA | Styleze ® | 5.00 | 5.00 | ISP |
| Acrylates | CC-10 | | | |
| Copolymer | | | | |
| Quaternium-26 | Ceraphyl ® | 1.00 | 1.00 | ISP |
| | 65 | | | |
| Panthenol | Ritapan DL | 0.15 | 0.15 | RITA |
| Propylene Glycol | Liquid | 0.50 | 0.50 | ISP |
| (and) Diazolidinyl | Germall ® | | | |
| Urea (and) | Plus | | | |
| Iodopropynyl | | | | |
| Butylcarbamate | | | | |
| | Hydrolysate of | 1.00 | 1.00 | ISP |
| | example 1 | | | |
| Total | | 100.00 | 100.00 | |

Disperse the Natrosol 250HHR and the Disodium EDTA in the water with stiffing. Heat to 50-60° C. and stir until a uniform appearance is obtained. Add the Styleze® Cc-10 and stir until a uniform appearance is obtained. Allow to cool to ambient temperature and add the ingredients in the order listed while stirring until a uniform appearance is obtained after each addition.

3—Non-Aerosol Treatment Foam:

Apply the product to the wet scalp. Massage to spread the product uniformly. Strengthens hair vitality and health and promotes a long styling hold, especially in a damp atmosphere.

| | | Formulation | | |
|---|---|---|---|---|
| | | 1 | 2 | |
| | | % by | % by | |
| INCI Name | Trade name | weight | weight | Supplier |
| Water | | Q.S. | Q.S. | |
| PEG-45M | POLYOX N-750 | 0.075 | 0.075 | Dow |
| Polyquaternium-55 | Styleze ® W | 5.00 | 5.00 | ISP |
| (20%) | | | | |
| Propylene Glycol | Liquid | 0.50 | 0.50 | ISP |
| (and) Diazolidinyl | Germall ® Plus | | | |
| Urea (and) | | | | |
| Iodopropynyl | | | | |
| Butylcarbamate | | | | |
| PEG/PPG-25/25 | SiTec ™ DMC | 1.00 | — | ISP |
| Dimethicone | 6031 | | | |
| Palmitamido- | Varisoft PATC | — | 0.80 | Degussa |
| propyltrimonium | | | | |
| Chloride | | | | |
| | Hydrolysate of | 1.00 | 1.00 | ISP |
| | example 1 | | | |
| Total | | 100.00 | 100.00 | |

Put water in a suitable container and stir vigorously to create a vortex. Disperse the Polyox into the vortex and stir until completely dissolved. Add the Styleze W-20 and stir until a uniform appearance is obtained. Next, add the ingredients in the order listed while stirring until a uniform appearance is obtained after each addition.

1. Cosmetic use of a composition comprising at least one yeast (Saccharomyces cerevisiae) peptide hydrolysate, obtained by cells disruption and elimination of most of the insoluble membrane components, as an active agent for strengthening hair and improving hair health.

2. A use according to claim 1, characterized in that the peptide hydrolysate has a protein concentration of 30% to 70% of the total weight of the dry extract, and more specifically 40% to 50% of the total weight of the dry extract.

3. A use according to claim 1, characterized in that the peptide hydrolysate is dissolved in one or more physiologically acceptable solvents, such as water, glycerol, ethanol, propanediol, butylene glycol, dipropylene glycerol, ethoxylated or propoxylated diglycols, cyclic polyhydric alcohols, or any mixture of these solvents.

4. A use according to claim 1, characterized in that the peptide hydrolysate is used in a quantity of 0.001% to 5% of the total weight of the composition, and preferably in a quantity of 0.01% to 1% of the total weight of the composition.

5. A use according to claim 1, characterized in that the composition is in a form suitable for topical application having a physiologically acceptable medium.

6. A use according to claim 1, characterized in that the composition furthermore comprises at least one other active agent which protects or improves hair growth and/or health.

7. A use according to claim 1 to counteract alopecia.

8. A cosmetic use of a peptide hydrolysate as an active agent as defined in claim 1, to strengthen the hair follicle structures.

9. A use according to claim 8, characterized in that the active agent increases the expression of keratin 14 and keratin K17 in the external sheath cells of the hair follicle.

10. A use according to claim 8, characterized in that the active agent increases the expression of collagen IV and protein CD34 in the wall of the hair follicle blood vessels.

11. A use according to claim 8, characterized in that the active agent increases the expression of collagen I in the conjunctive tissue sheath and dermal papilla of the hair follicle.

12. A method of cosmetic treatment intended to restore and/or stimulate hair growth, or counteract hair loss, characterized in that a composition as defined in claim 1 is applied topically to the area being treated.

13. A method of cosmetic treatment intended to restore and/or stimulate eyelash growth, or counteract eyelash loss, characterized in that a composition as defined in claim 1 is applied topically to the area being treated.

14. A use of a yeast peptide hydrolysate as defined in claim 1 as an active agent capable of strengthening and protecting the hair follicle, for the preparation of a dermo-pharmaceutical composition intended to stimulate hair growth or prevent hair loss related to a pathological condition.

15. A use according to the claim 14, characterized in that the pathological conditions are alopecia areata, the side effects of drug treatments, and certain infections or inflammations of the scalp.
